(19) United States
(12) Patent Application Publication
(10) Pub. No.: US 2020/0407407 A1
(43) Pub. Date: Dec. 31, 2020

(54) Oral Gene Carrier And Use Thereof

(71) Applicant: KB BIOMED INC., Chungcheongbuk-do (KR)

(72) Inventors: Yong Kyu LEE, Chungcheongbuk-do (KR); Seung Bin CHA, Gyeonggi-do (KR); Sung Hun KANG, Chungcheongbuk-do (KR); Sun Hwa LEE, Daejeon (KR)

(21) Appl. No.: 16/956,784

(22) PCT Filed: Dec. 21, 2018
(86) PCT No.: PCT/KR2018/016406

§ 371 Date: Aug. 20, 2020

(30) Foreign Application Priority Data
Dec. 22, 2017 (KR) 10-2017-0177676
Dec. 20, 2018 (KR) 10-2018-0166594

(51) Int. Cl. C07K 14/46; A61K 47/64; A61P 3/10; A61K 38/26

(52) U.S. Cl. CPC C07K 14/46; A61K 38/26; A61P 3/10; A61K 47/64

(57) ABSTRACT

The present invention relates to an orally-administered gene carrier, and more specifically, to an orally-administered gene carrier having cationic protamine connected to an immunoglobulin Fc region by an SMCC linker, the cationic protamine enabling the condensation of an anionic gene. The orally-administered gene carrier enables protamine, which is a protein having cationic properties, to bind to an Fc region and be condensed with a gene having anionic properties, and thus may effectively induce the in vivo expression of the gene, and when orally administered, may enable the gene to be transferred to the small intestine by protecting the gene from a degradation reaction resulting from an immune action of white blood cells and stomach acid, and may enable the half-life of the gene to be relatively long when the gene is expressed in the small intestine, and thus a potential for a long-term treatment effect has been confirmed.

### FIELD

The present invention relates to an orally-administered gene carrier, and more specifically, to an orally-administered gene carrier having cationic protamine connected to an immunoglobulin Fc region by an SMCC linker, the cationic protamine enabling the condensation of an anionic gene.

### BACKGROUND

An Fc region of an antibody serves to recruit immune leukocytes or serum complement molecules, thereby allowing damaged cells such as cancer cells or infected cells to be removed. The site on Fc between the Cγ2 and Cγ3 domains mediates the interaction with a neonatal receptor FcRn and the binding recirculates an intracellularly introduced antibody from the endosome to the bloodstream. This process is associated with the inhibition of kidney filtration due to the enormous size of a full-length molecule, thereby having an advantageous antibody serum half-life ranging from 1 to 3 weeks. Further, the binding of Fc to FcRn also plays an important role in antibody transport. Therefore, the Fc region plays an essential role in maintaining the prolonged serum persistence of an antibody because the antibody is circulated through an intracellular trafficking and recycling mechanism.

Meanwhile, although the concept of oral gene therapy has already been proved, non-viral gene delivery through an intestinal segment has a problem of low expression levels, which suffers from many difficulties. Furthermore, there remains a challenge of effectively controlling the degradation by intestinal enzymes, microorganisms, and digestive juices. However, a carrier preparation by an Fc receptor (FcRn) has the ability to pass through the intestinal epithelial cells, so that it is possible to solve the aforementioned problems due to absorption efficiency. In particular, circulation using the Fc receptor has the ability to circulate for the longest time as compared to other circulation methods, and has a half-life between 7 to 21 days under human conditions, so that it is possible to have better effects than other IgG types.

Accordingly, in many ongoing clinical studies, a lot of effort has been made to introduce mutations into an Fc region in order to increase the half-life of the antibody, or to develop a next-generation anticancer antibody therapeutic agent or an anticancer protein therapeutic agent through an Fc domain into which mutations are introduced in order to maximize an antibody-dependent cellular cytotoxicity (ADCC) effect (Korean Patent Application Laid-Open No. 10-2017-0106258). However, the results are still incomplete.

### SUMMARY

### Technical Problem

The present invention has been devised to solve the problems as described above, and as a result of intensive studies on a carrier for efficiently delivering a gene into in vivo cells, the present inventors confirmed that a gene carrier which enables protamine having cationic properties, to be linked to an immunoglobulin Fc region by SMCC in order to effectively condense a gene having anionic properties was stable against pH and systemic enzymes, and confirmed its usability as an orally-administered gene carrier, thereby completing the present invention based on this.

Thus, an object of the present invention is to provide an orally-administered gene carrier including: protamine which binds to a target gene;

an immunoglobulin Fc region; and

a linker which links the protamine and the immunoglobulin Fc region.

Further, another object of the present invention is to provide a method for preparing the gene carrier.

In addition, still another object of the present invention is to provide a pharmaceutical composition for preventing or treating diabetes mellitus, the pharmaceutical composition including the gene carrier and a GLP-1 gene bound to the carrier as active ingredients.

However, technical problems to be achieved by the present invention are not limited to the aforementioned problems, and other problems that are not mentioned may be clearly understood by those skilled in the art from the following description.

### Technical Solution

To achieve the objects as described above, the present invention provides an orally-administered gene carrier including:

protamine which binds to a target gene;

an immunoglobulin Fc region; and

a linker which links the protamine and the immunoglobulin Fc region.

As an exemplary embodiment of the present invention, the immunoglobulin Fc gene may include an amino acid sequence of SEQ ID NO: 1.

As another exemplary embodiment of the present invention, the immunoglobulin Fc region may include a base sequence of SEQ ID NO: 2.

As still another exemplary embodiment of the present invention, the immunoglobulin Fc region may be derived from any one selected from the group consisting of IgG, IgA, IgD, IgE, and IgM.

As yet another exemplary embodiment of the present invention, the immunoglobulin Fc region may be derived from IgG.

As yet another exemplary embodiment of the present invention, the linker may be succinimidyl 4-(N-maleimido-methyl)cyclohexane-1-carboxylate) (SMCC).

As yet another exemplary embodiment of the present invention, the gene carrier may be prepared by being mixed with the target gene at a weight ratio (w/w) of 1:5 to 50:1.

Further, the present invention provides a method for preparing the gene carrier, the method comprising the following steps:

(a) preparing a protamine-succinimidyl 4-(N-maleimido-methyl)cyclohexane-1-carboxylate) (SMCC) solution by adding a protamine solution to a SMMC solution;

(b) preparing a protamine-SMCC-Fc solution by adding a Cys-Fc solution to the protamine-SMCC solution; and

(c) obtaining a gene carrier by freeze-drying the prepared protamine-SMCC-Fc solution.

In addition, the present invention provides a pharmaceutical composition for preventing or treating diabetes mellitus, the composition comprising the gene carrier and a glucagon like peptide-1 (GLP-1) gene bound to the carrier as active ingredients.

As an exemplary embodiment of the present invention, the diabetes mellitus may be type 2 diabetes mellitus.

As another exemplary embodiment of the present invention, the GLP-1 gene may include a base sequence of SEQ ID NO: 3.

In addition, the present invention provides a method for preventing or treating diabetes mellitus, the method including: administering the pharmaceutical composition to an individual.

Furthermore, the present invention provides a use of the pharmaceutical composition for preventing or treating diabetes mellitus.

### Advantageous Effects

The orally-administered gene carrier, according to the present invention, enables protamine, which is a protein having cationic properties, to bind to an Fc region and be condensed with a gene having anionic properties, and thus can effectively induce the in vivo expression of the gene, and when orally administered, can enable the gene to be delivered to the small intestine by protecting the gene from a degradation reaction resulting from an immune action of white blood cells and stomach acid, and can enable the half-life of the gene to be relatively long when the gene is expressed in the small intestine, and thus a potential for a long-term treatment effect has been confirmed. Thus, the gene carrier, according to the present invention, is expected to be usefully employed as an orally-administered carrier for various genes.

### BRIEF DESCRIPTION OF DRAWINGS

FIG. 1A illustrates NMR analysis results in order to confirm physical characteristics of protamine-SMCC-Fc according to the present invention.

FIG. 1B illustrates FT-IR analysis results of protamine-SMCC-Fc.

FIG. 1C illustrates the results of confirming whether protamine-SMCC and Fc regions are conjugated using SDS-PAGE.

FIG. 2A is a set of dynamic light scattering (DLS) analysis results for confirming particle sizes of protamine-SMCC-Fc.

FIG. 2B illustrates SEM imaging results of protamine-SMCC-Fc.

FIG. 2C illustrates results of measuring the size (DLS analysis) and zeta potential of each complex prepared by mixing DNA and protamine-SMCC-Fc at various wt % (w/w) ratios.

FIG. 3 illustrates results of verifying the acid stability of a GLP-1 and protamine-SMCC-Fc complex (DNA+Protamine-SMCC-Fc) under various pH conditions.

FIG. 4A illustrates SEM images of a DNA/protamine-SMCC-Fc complex.

FIG. 4B illustrates AFM analysis results of a DNA/protamine-SMCC-Fc complex.

FIG. 5 illustrates results of subjecting a complex prepared using GLP-1 DNA and protamine-SMCC-Fc at various ratios to agarose gel electrophoresis in order to confirm the conjugation of a DNA/protamine-SMCC-Fc complex.

FIG. 6 illustrates serum stability and DNase analysis results in order to confirm the ability of the protamine-SMCC-Fc gene carrier according to the present invention to protect genes from serum and enzymatic degradation.

FIG. 7 illustrates FcRn-mediated cellular uptake experimental results of protamine-SMCC-Fc of the protamine-SMCC-Fc gene carrier according to the present invention in FcRn positive cells (HT29) and negative cells (KB), respectively.

FIG. 8 illustrates results of measuring cell viability after HT-29 cells are treated with a protamine-SMCC-Fc gene carrier at each concentration.

FIG. 9 illustrates results of confirming the cell permeability of the protamine-SMCC-Fc gene carrier.

FIG. 10 illustrates results of confirming the condensation of GLP-1 for the GLP-1/protamine-SMCC-Fc complex.

FIG. 11 illustrates results of in vivo experiments in which non-fasting blood sugar is measured while orally administering the GLP-1-protamine-SMCC-Fc complex to an animal model at 4-day intervals.

### DETAILED DESCRIPTION

Hereinafter, the present invention will be described in detail.

As a result of intensive studies on a carrier for efficiently delivering a gene into in vivo cells, the present inventors confirmed that a gene carrier which enables protamine which is a protein having cationic properties, to bind to an Fc region in order to efficiently condense a gene having anionic properties was stable against pH and systemic enzymes, and confirmed its usability as an orally-administered carrier, thereby completing the present invention.

Thus, the present invention provides an orally-administered gene carrier including: protamine which binds to a target gene;

an immunoglobulin Fc region; and

a linker which links the protamine and the immunoglobulin Fc region.

As used herein, the term “protamine” refers to a natural cationic protein rich in arginine, and among the testes of animals, is abundant in the sperm nuclei of fish especially salmon, and is known as a protein which is involved in the expression of genetic information by association or dissociation with DNA like histones. Typically, the molecular weight of protamine extracted from the sperm nuclei of fish is about 4,000 to 10,000, and 70% or more of the constituent amino acids are present as arginine, but the protamine used in the present invention is not limited thereto.

As used herein, the term “immunoglobulin Fc region” refers to heavy chain constant region 2 (CH2) and heavy chain constant region 3 (CH3) portions, excluding heavy and light chain variable regions, a heavy chain constant region 1 (CH1) and a light chain constant region 1 (CL1) of an immunoglobulin, and also includes a hinge portion in the heavy chain constant region. Further, the immunoglobulin Fc region of the present invention may be an extended Fc region including a part or the entirety of the heavy chain constant region 1 (CH1) and/or the light chain constant region 1 (CL1), excluding the heavy and light chain variable regions of the immunoglobulin, as long as the immunoglobulin Fc region of the present invention has substantially the same effect as or an improved effect compared to that of a natural type. In addition, the immunoglobulin Fc region may also be a region in which a significantly long partial amino acid sequence corresponding to CH2 and/or CH3 is removed.

Furthermore, the immunoglobulin Fc region of the present invention includes not only a natural-type amino acid sequence but also a sequence derivative (mutant) thereof. An amino acid sequence derivative means that one or more amino acid residues in a natural amino acid sequence have different sequences due to deletion, insertion, non-conservative or conservative substitution, or a combination thereof.

In the present invention, the immunoglobulin Fc region may include an amino acid sequence of SEQ ID NO: 1, and a gene encoding the same may include a base sequence of SEQ ID NO: 2. In this case, the orally-administered gene carrier may include an amino acid sequence or a base sequence, which has a sequence homology of 70% or more, preferably 80% or more, more preferably 90% or more, even more preferably 95% or more, and most preferably 98% or more with the amino acid sequence represented by SEQ ID NO: 1 or the base sequence represented by SEQ ID NO: 2, respectively.

Furthermore, these Fc regions may be obtained from a natural type isolated in vivo from an animal such as a human, a cow, a goat, a pig, a mouse, a rabbit, a hamster, a rat, or a guinea pig, and may be a recombinant type obtained from transformed animal cells or microorganisms, or a derivative thereof. Here, the method of obtaining an Fc region from a natural type may be a method of obtaining an Fc region by isolating an entire immunoglobulin from a human or animal organism and then treating the entire immunoglobulin with a proteolytic enzyme. When the immunoglobulin is treated with papain, the immunoglobulin is cleaved into Fab and Fc, and when the immunoglobulin is treated with pepsin, the immunoglobulin is cleaved into pF′c and F(ab)2. Fc or pF′c may be isolated from the cleaved portions using size exclusion chromatography, and the like. In the present invention, the immunoglobulin Fc region is a recombinant type immunoglobulin Fc region, preferably, a human-derived Fc region obtained from a microorganism.

Further, the immunoglobulin Fc region may be a natural sugar chain, an increased sugar chain compared to the natural type, a decreased sugar chain compared to the natural type, or a form in which a sugar chain is removed. In order to increase/decrease or remove the immunoglobulin Fc sugar chains, a typical method such as a chemical method, an enzymatic method, and a genetic engineering method using microorganisms may be used. In this case, the immunoglobulin Fc region in which the sugar chain is removed from Fc does not cause unnecessary immune reactions in vivo, because the binding power to a complement (c1q) is remarkably reduced, and antibody-dependent cytotoxicity or complement-dependent cytotoxicity is reduced or removed. In this regard, a form which is more consistent with the intended purpose as a drug carrier may be said to be an immunoglobulin Fc region in which the sugar chain has been removed or deglycosylated.

In addition, the immunoglobulin Fc region may be an Fc region derived from IgG, IgA, IgD, IgE, IgM, or a combination thereof or a hybrid thereof, and is most preferably derived from IgG known to improve the half-life of a ligand-binding protein most abundant in human blood.

In the present invention, in order to effectively condense a gene having anionic properties, protamine having a cationic property was bound to an immunoglobulin Fc region, and in this case, a linker for binding the protamine and the Fc region may be sulfosuccinimidyl 4-(N-maleimido-methyl)cyclohexane-1-carboxylate) (sulfo-SMCC), but is not limited thereto, and any linker may be used without any limitation as long as the linker has a property of selectively reacting with both an amine group and a thiol group.

Thus, as another aspect of the present invention, the present invention provides a method for preparing the gene carrier, the method comprising the following steps:

(a) preparing a protamine-succinimidyl 4-(N-maleimido-methyl)cyclohexane-1-carboxylate (SMCC) solution by adding a protamine solution to a SMMC solution;

(b) preparing a protamine-SMCC-Fc solution by adding a Cys-Fc solution to the protamine-SMCC solution; and

(c) obtaining a gene carrier by freeze-drying the prepared protamine-SMCC-Fc solution.

In exemplary embodiments of the present invention, in vitro and in vivo analyses were performed in order to confirm the characteristics of a gene carrier prepared by the method and verify its usability.

In an exemplary embodiment, a gene carrier of protamine-SMCC-Fc in which an immunoglobulin Fc region and protamine were linked was synthesized using SMCC as the linker (see Example 1).

In another exemplary embodiment of the present invention, in order to confirm physical characteristics of the gene carrier of protamine-SMCC-Fc, NMR and FT-IR experiments were performed, an optimal conjugation ratio was confirmed through a TNBSA analysis, and it was confirmed by SDS-PAGE electrophoresis whether protamine-SMCC-Fc was conjugated (see Example 2).

In still another exemplary embodiment of the present invention, the conjugation state of the protamine-SMCC-Fc with DNA was specifically confirmed, it was confirmed that the protamine-SMCC-Fc was conjugated with DNA at a ratio of the protamine-SMCC-Fc:DNA of 1:1 or more, and it was confirmed that the complex was stable under various pH conditions (see Example 3). Furthermore, in order to confirm the efficient in vivo delivery effect of a gene, the stability and cytotoxicity to serum were first confirmed, and a remarkably high uptake effect in HT-29 cells which are FcRn-positive cells was confirmed by confirming cellular uptake.

Further, for the cell permeability of the gene carrier, the ability of the gene carrier to permeate cells was confirmed by performing a cell membrane permeability experiment of a HT-29 monolayer, and finally, the gene condensation effect was specifically confirmed (see Example 4).

In addition, in yet another exemplary embodiment of the present invention, as a result of administering a GLP-1-protamine-SMCC-Fc complex to an animal model at an interval of 4 days and measuring non-fasting blood sugar through an in vivo experiment, it was confirmed that the blood sugar was restored to a normal level unlike that of a control (see Example 5).

Through the results, the present inventors confirmed that the orally-administered gene carrier according to the present invention is stable against pH and enzymes, and the carrier is absorbed in various organs, and the uptake rate is high in intestinal organs, so that protamine-SMCC-Fc has excellent binding power to various organs, and thus based on this, is expected to exhibit an efficient ability when used as a gene carrier. Furthermore, it is suggested that the orally-administered gene carrier according to the present invention can be applied as a carrier of various genes in the future.

Thus, as still another aspect of the present invention, the present invention provides a pharmaceutical composition for preventing or treating diabetes mellitus, the composition comprising the gene carrier and a glucagon like peptide-1 (GLP-1) gene bound to the carrier as active ingredients.

In the present invention, the diabetes mellitus may be type 2 diabetes mellitus, but is not limited thereto.

The GLP-1 may include a base sequence of SEQ ID NO: 3, and in this case, the GLP-1 may include a base sequence having a sequence homology of 70% or more, preferably 80% or more, more preferably 90% or more, even more preferably 95% or more, and most preferably 98% or more with the base sequence represented by SEQ ID NO: 3.

As used herein, the term “prevention” refers to all actions that suppress diabetes mellitus or delay the onset of the diabetes mellitus by administering the pharmaceutical composition according to the present invention.

As used herein, the term “treatment” refers to all actions that ameliorate or beneficially change symptoms caused by diabetes mellitus by administering the pharmaceutical composition according to the present invention.

The pharmaceutical composition according to the present invention includes the gene carrier and the GLP-1 gene as active ingredients, and may further include a pharmaceutically acceptable carrier. The pharmaceutically acceptable carrier is typically used in formulation, and includes saline, sterile water, Ringer's solution, buffered saline, cyclodextrin, a dextrose solution, a maltodextrin solution, glycerol, ethanol, liposomes, and the like, but is not limited thereto, and may further include other typical additives such as an antioxidant and a buffer, if necessary. Further, the oral composition according to the present invention may be formulated into an injectable formulation, such as an aqueous solution, a suspension, and an emulsion, a pill, a capsule, a granule, or a tablet by additionally adding a diluent, a dispersant, a surfactant, a binder, a lubricant, and the like. With regard to suitable pharmaceutically acceptable carriers and formulations, the composition may be preferably formulated according to each ingredient by using the method disclosed in Remington's literature. The pharmaceutical composition of the present invention is not particularly limited in formulation, but may be formulated into an injection, an inhalant, an external preparation for skin, or the like.

The pharmaceutical composition of the present invention may be orally administered or may be parenterally administered (for example, applied intravenously, subcutaneously, intraperitoneally, or locally), but may be preferably orally administered, and the administration dose may vary depending on a patient's condition and body weight, severity of disease, drug form, and administration route and period according to the target method, but the administration dose may be properly selected by those skilled in the art.

The pharmaceutical composition of the present invention is administered in a pharmaceutically effective amount. As used herein, the “pharmaceutically effective amount” refers to an amount sufficient to treat or diagnose diseases at a reasonable benefit/risk ratio applicable to medical treatment or diagnosis, and an effective dosage level may be determined according to factors including the type of disease of patients, the severity of disease, the activity of drugs, sensitivity to drugs, administration time, administration route, excretion rate, treatment period, and simultaneously used drugs, and other factors well known in the medical field. The pharmaceutical composition according to the present invention may be administered as an individual therapeutic agent or in combination with other therapeutic agents, may be administered sequentially or simultaneously with therapeutic agents in the related art, and may be administered in a single dose or multiple doses. It is important to administer the composition in a minimum amount that can obtain the maximum effect without any side effects, in consideration of all the aforementioned factors, and this amount may be easily determined by those skilled in the art.

Specifically, an effective amount of the pharmaceutical composition of the present invention may vary depending on the age, sex, condition, and body weight of a patient, the absorption of the active ingredients in the body, inert rate and excretion rate, disease type, and the drugs used in combination, and in general, 0.001 to 150 mg, preferably 0.001 to 100 mg of the pharmaceutical composition of the present invention per 1 kg of a body weight may be administered daily or every other day or may be dividedly administered once to three times a day. However, since the effective amount may be increased or decreased depending on the administration route, the severity of obesity, gender, body weight, age, and the like, the dosage is not intended to limit the scope of the present invention in any way.

As yet another aspect of the present invention, the present invention provides a method for preventing or treating diabetes mellitus, the method including: administering the pharmaceutical composition to an individual.

As used herein, the “individual” refers to a subject in need of treatment of a disease, and more specifically, refers to a mammal such as a human or a non-human primate, a mouse, a rat, a dog, a cat, a horse, and a cow.

Further, the present invention provides a use of the pharmaceutical composition for preventing or treating diabetes mellitus.

Hereinafter, preferred examples for helping the understanding of the present invention will be suggested. However, the following examples are provided only to more easily understand the present invention, and the contents of the present invention are not limited by the following examples.

### EXAMPLES

### Examples 1: Preparation of Gene Carrier

1-1. Synthesis of Protamine-SMCC

Protamine (5.1 kD) (1 mol) was dissolved in dH2O (5 mg/mL), the resulting solution was stirred for 30 minutes, and then pH was adjusted to 7.5 in order to activate NH₂ groups. Meanwhile, sulfo-SMCC (2 mol) was dissolved in a solution (5 mg/mL) in which 300 uL of DMSO and 700 uL of dH2O were mixed, and then the Sulfo-SMCC solution was added dropwise to the solution of protamine prepared above, and in this case, the resulting solution was continuously stirred at room temperature for 24 hours. Thereafter, the reaction mixture was dialyzed against distilled water for 2 days (MWCO:3.5-5 kD) and lyophilized to obtain protamine-SMCC.

1-2. Preparation of Protamine-SMCC-Fc

First, protamine-SMCC was dissolved in PBS at a pH of 6.5 to 7.4, the resulting solution was made to have a concentration of 3 mg/mL while being stirred for 30 minutes, and then after a solution having a concentration of 16 mg/ml was produced by separately dissolving Cys-Fc in PBS (1 mol), the solution was added to the protamine-SMCC solution in a dropwise manner while stirring the solution at room temperature for 1 hour. Thereafter, the resulting product was cultured at 4° C. overnight (12 hours) without separate stirring, and then dialyzed with dH2O for 24 hours and lyophilized to obtain protamine-SMCC-Fc.

### Example 2. Confirmation of Physical Characteristics of Protamine SMCC-Fc

2-1. NMR and FT-IR Analysis

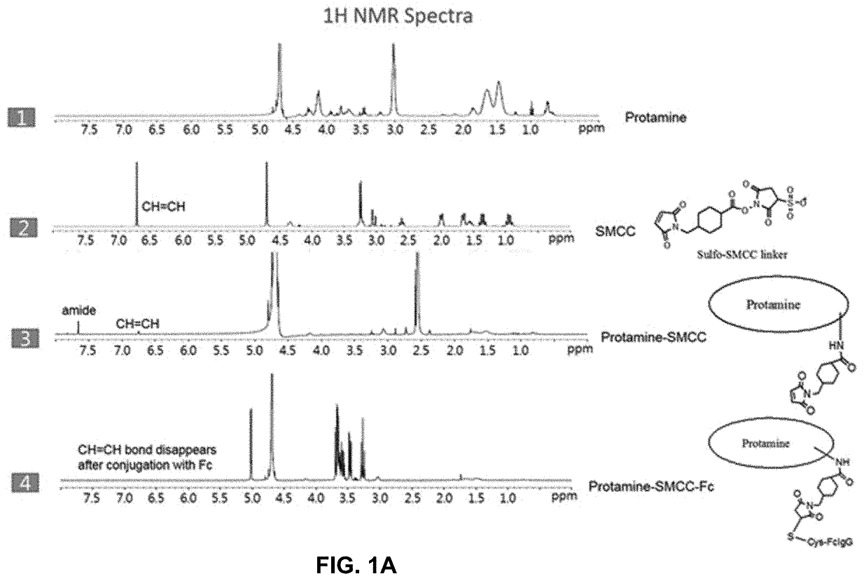

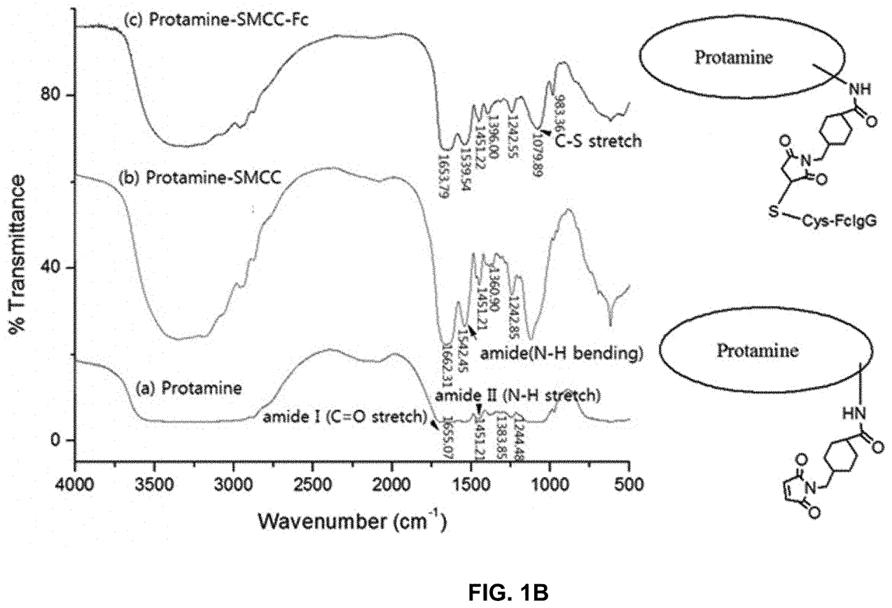

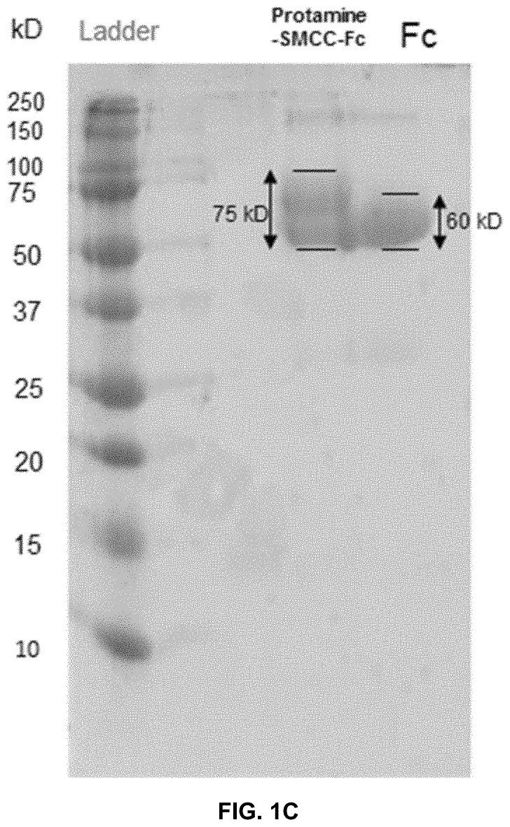

In order to confirm physical characteristics of the protamine-SMCC-Fc obtained from Example 1, NMR and FT-IR experiments were performed, and the results thereof are shown in FIGS. 1A and 1B. More specifically, FIG. 1A illustrates NMR analysis spectrum results of protamine (No. 1), SMCC (No. 2), protamine-SMCC (No. 3), and protamine-SMCC-Fc (No. 4), and FIG. 1B illustrates FT-IR analysis results of protamine (a), protamine-SMCC (b), and protamine-SMCC-Fc (c).

2-2. TNBSA Analysis

In order to confirm an optimal conjugation ratio of protamine and SMCC, a 2,4,6-trinitrobenzene sulfonic acid (TNBSA) analysis was performed.

As a result, as shown in the following Table 1, it was confirmed that a ratio in which 0.4 nm SMCC was conjugated with 1 nm protamine was an optimal ratio, and specifically, when a feed mole ratio of protamine-SMCC was 1:2 and 1:3, there was no difference in conjugation ratio, so that the mole ratio was optimized at 1:2.

TABLE 1

| Feed mole ratio (Protamine: SMCC) | Conjugation ratio |
|---|---|
| 1:2 | 1:0.4 |
| 1:3 | 1:0.4 |

2-3. SDS-PAGE Electrophoresis

Further, as a result of verifying wither protamine-SMCC-Fc was conjugated through SDS-PAGE using 12% acrylamide gel, as illustrated in FIG. 1C, it was confirmed that the molecular weight (75 kDa) of a protamine-SMCC-Fc sample was shown to be larger than that of an Fc alone sample (60 kDa).

2-4. Particle Size Analysis

In order to confirm the particle size of the protamine-SMCC-Fc according to the present invention, a dynamic light scattering (DLS) analysis and a scanning electron microscope (SEM) image analysis were performed.

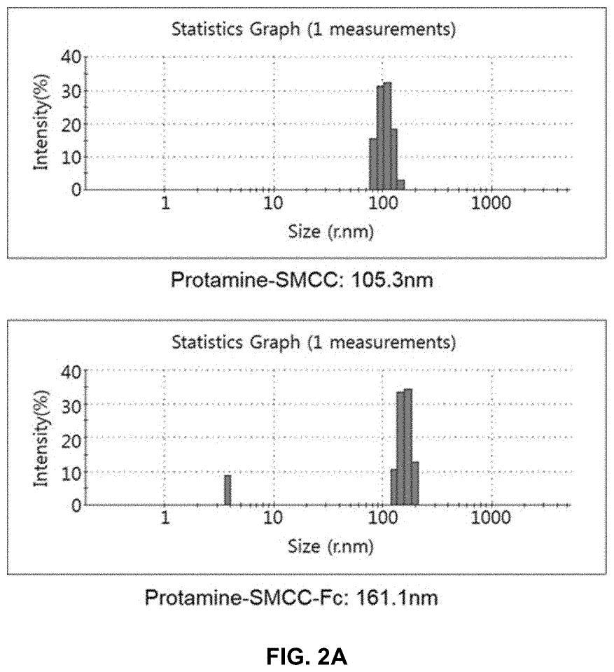

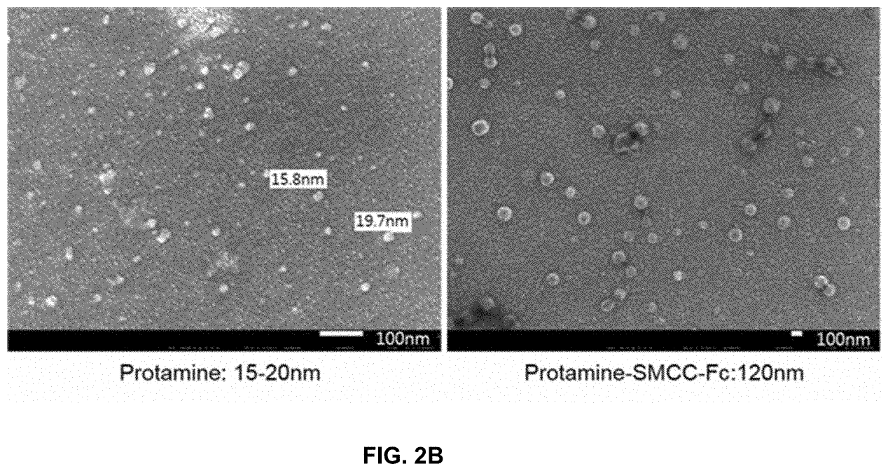

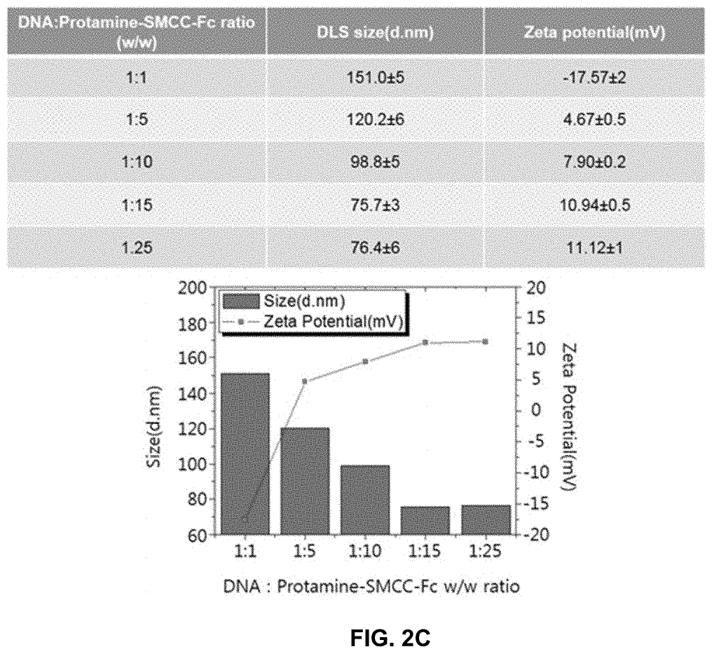

As a result, as illustrated in the DLS analysis result of FIG. 2A, it was confirmed that the size of the protamine-SMCC particle was 105.3 nm, whereas the size of protamine-SMCC-Fc was 161.1 nm, and the particle size was increased by Fc conjugation. In addition, as a result of an SEM imaging result, as illustrated in FIG. 2B, it was confirmed that the protamine-SMCC-Fc particles showed a monodisperse morphology, and the particle size was measured similarly to the DLS analysis results. As a result of the DLS and SEM analysis, the measured protamine-SMCC-Fc particle sizes are summarized and shown in the following Table 2.

TABLE 2

| name | DLS size | SEM size |
|---|---|---|
| Protamine-SMCC | 105.3 nm | 90 nm |
| Protamine-SMCC- | 161.1 nm | 120 nm |
| Fc | | |

### Example 3. Electrostatic Interaction Study

3-1. Measurement of Particle Size and Zeta Potential

Particle sizes were measured by DLS analysis of complexes in which the GLP1 DNA and the protamine-SMCC-Fc carrier were mixed at various ratios (1:1, 1:5, 1:10, 1:15, and 1:25), and Zeta potential analysis was performed.

As a result, as illustrated in FIG. 2C, when the ratio of a GLP1 DNA to a protamine-SMCC-Fc carrier is 1:15 or more, the particle size is not decreased even though the Zeta potential is similar. So that it was confirmed that the DNA and the carrier were completely compacted to form a complex.

3-2. DSC: pH Stability Analysis

Next, when a drug is orally administered, the pH is different at each organ in the body, so that it was intended to verify the stability of the GLP1 and protamine-SMCC-Fc complex against various pH environments. For this purpose, each complex prepared by mixing the GLP1 DNA and the protamine-SMCC-Fc carrier at various ratios was placed under pH 7.4, 5.6, and 1.2 conditions, and the particle size was measured by DLS analysis at each time point (0, 3, 6, and 24 hours).

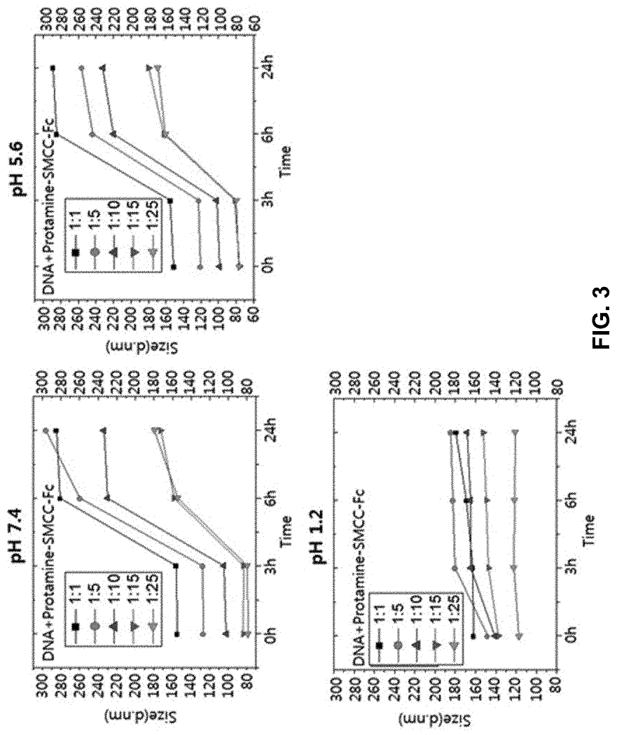

As a result, as illustrated in FIG. 3, it was shown that the complex was stable at all of pH 7.4, 5.5, and 1.2, the particle size was decreased until a ratio of 1:15, the sizes were similar in the complex at a ratio of 1:25, and thus it was confirmed once again that the optimal ratio of the GLP1 DNA to the protamine-SMCC-Fc carrier was 1:15.

3-3. SEM and AFM Analysis

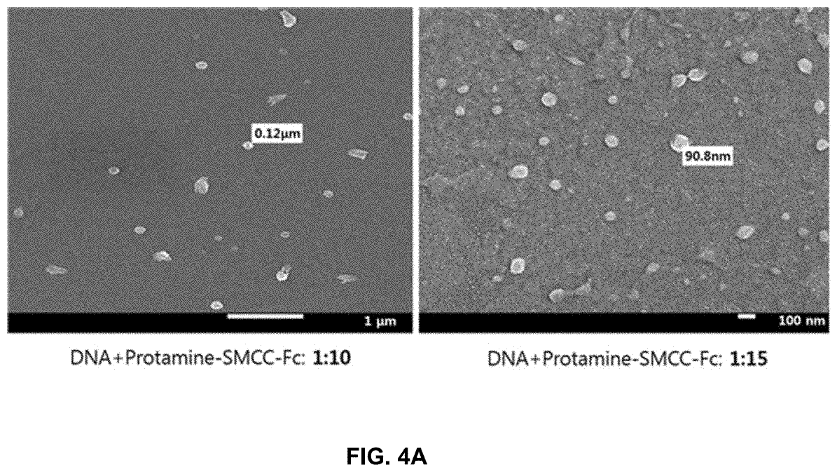

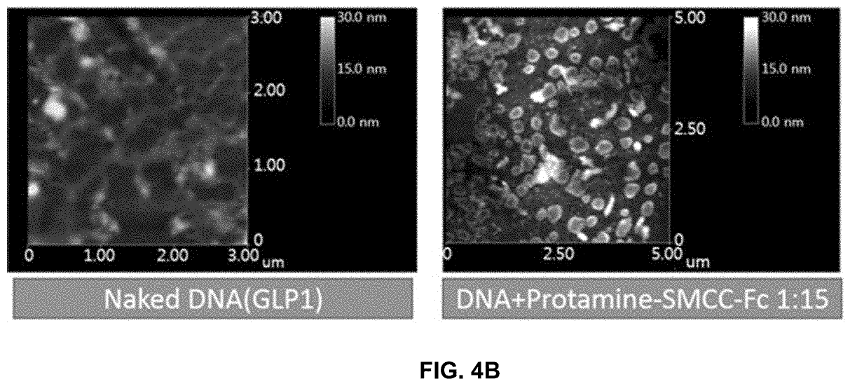

First, in order to confirm the morphology of a complex in which the GLP-1 DNA and the protamine-SMCC-Fc carrier were mixed, the complexes were prepared at a ratio of 1:10 and 1:15, respectively, and then an SEM image analysis was performed, and the results thereof are shown in FIG. 4.

Further, as a result of an atomic force microscope (AFM) analysis, as can be seen in FIG. 4B, compared to the case of naked DNA, it was confirmed once again that a complex prepared at a ratio of DNA:protamine-SMCC-Fc of 1:15 showed a binding morphology similar to a spherical chain, and the ratio was an optimal binding ratio.

3-4. Agarose Gel Electrophoresis

It was intended to confirm whether a complex was formed by subjecting the complexes in which the GLP-1 DNA and the protamine-SMCC-FC carrier were mixed at various ratios to agarose gel electrophoresis. For this purpose, 1% agarose gel electrophoresis was performed using GLP-1 DNA alone, the complexes prepared at various ratios (1:1, 1:2, 1:5, 1:10, 1:12, 1:15, 1:25, and 1:30), and an Fc+GLP-1 DNA sample.

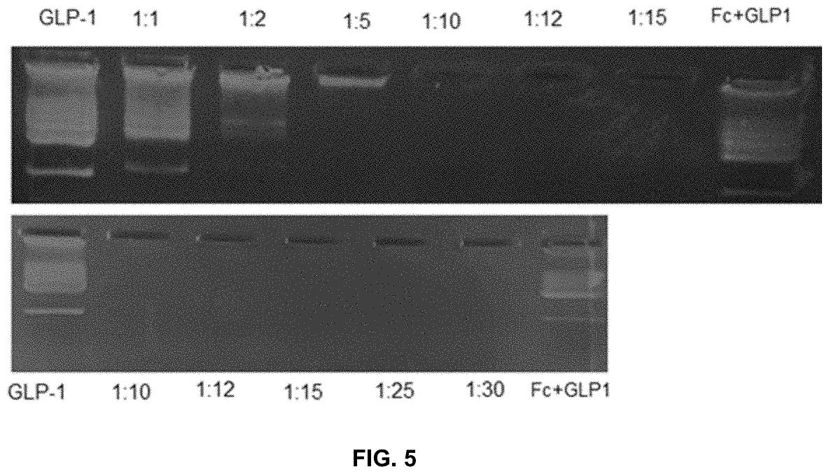

As a result, as illustrated in FIG. 5, it could be confirmed that, from a ratio of GLP-1:protamine-SMCC-Fc of 1:10, the band disappeared, and through this, it was confirmed that GLP-1 compactly bound to protamine-SMCC-Fc to form a complex.

### Example 4. In Vitro Study

4-1. Serum Stability Test and DNase Analysis

Serum stability and DNase analysis were performed in order to verify the ability of the orally-administered gene carrier according to the present invention to protect genes against serum and enzymatic degradation.

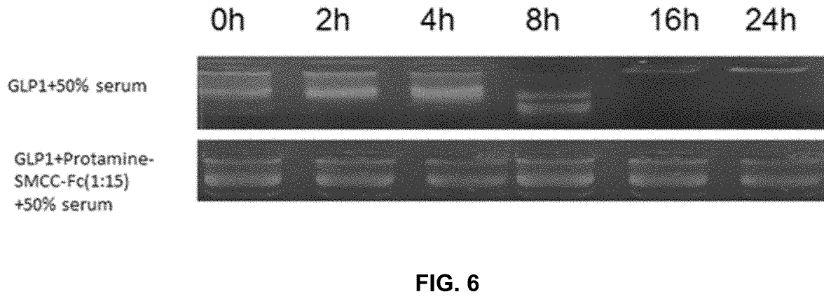

As a result, as illustrated in FIG. 6, it was confirmed that a GLP-1 gene alone began to be degraded in serum after 8 hours, whereas in the case of GLP-1+protamine-SMCC-Fc, protamine-SMCC-Fc protected the gene from serum degradation until 24 hours.

4-2. FcRn-Mediated Cellular Uptake Study

In order to confirm the interaction between FcRn and Fc and the uptake in FcRn positive cells, a study on cellular uptake of protamine-SMCC-Fc in HT-29 (FcRn+) and KB (FcRn−) cells was performed through confocal microscopy.

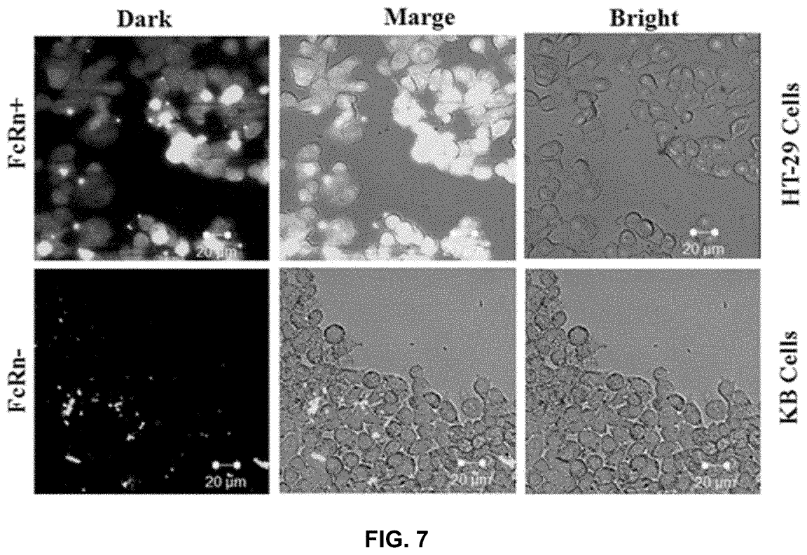

As a result, as illustrated in FIG. 7, it was confirmed that the protamine-SMCC-Fc showed remarkably high cellular uptake in FcRn positive HT-29 cells compared to KB cells.

4-3. Confirmation of Cytotoxicity

Next, in order to verify whether toxicity was caused by protamine-SMCC-Fc, HT-29 cells were treated with protamine-SMCC-Fc at various concentrations (5, 10, 25, 50, and 100 ug/ml), and after 24 hours and 72 hours, respectively, cell viability was measured by an MTT assay.

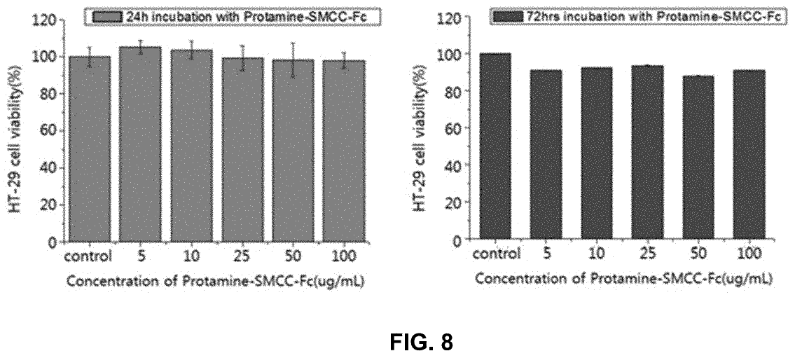

As a result, as illustrated in FIG. 8, it was confirmed that cell viability was maintained at high levels at all the concentrations until 72 hours compared to a control, so that it was confirmed that the gene carrier did not cause cytotoxicity in cells.

4-4. Confirmation of Epithelial Transport of Fc

In order to confirm the cell permeability of the gene carrier according to the present invention, HT-29 cells were cultured at 3×10⁵ cells/well/5.5 mL for 7 days and collected, and the medium was replaced with HBSS+0.05% BSA+10 mM MES pH 6.0 (apical chamber) and 10 mM HEPES, pH 7.4 (basolateral chamber), respectively. The basolateral portion was treated with different samples at different time intervals for fluorescence analysis, and then collected.

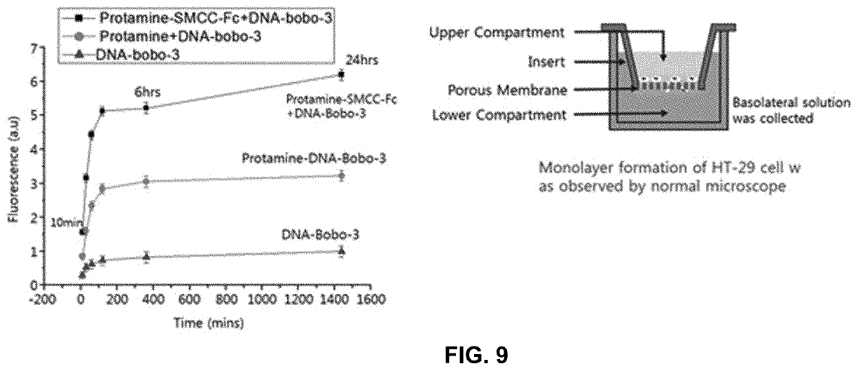

As a result, as illustrated in FIG. 9, it was confirmed that after 24 hours, cellular uptake was increased 5-fold in the gene carrier compared to the DNA alone.

4-5. GLP-1 Condensation Analysis

In order to confirm GLP-1 condensation for the GLP-1/protamine-SMCC-Fc complex according to the present invention, an EtBr displacement assay was performed.

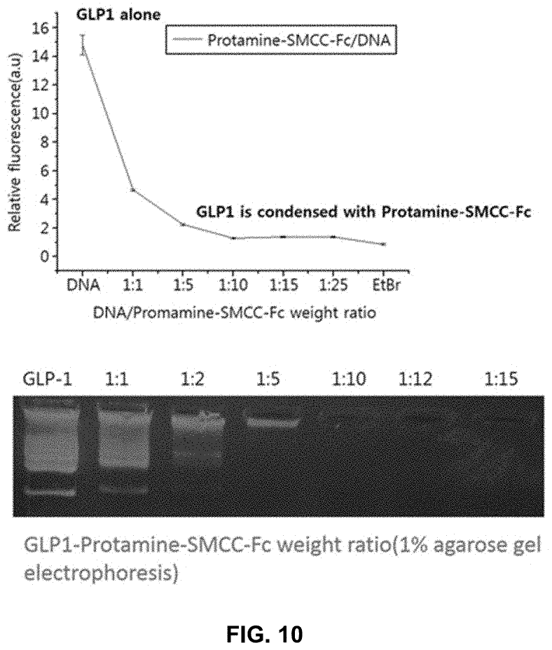

As a result, as illustrated in FIG. 10, it was confirmed that GLP-1 alone showed higher fluorescence than the GLP-1/protamine-SMCC-Fc complexes at different ratios, which is a result showing that GLP-1 was completely condensed by protamine-SMCC-Fc. From the above result, it was confirmed that the gene carrier according to the present invention was absorbed in various organs and the uptake rate was high in the intestinal organs, so that protamine-SMCC-Fc has excellent binding power to various organs, and thus, is expected to exhibit an efficient ability when used as a gene carrier. Furthermore, it was confirmed that protamine-SMCC-Fc could be applied as various gene carriers in the future.

### Example 5. In Vivo Study

In addition to the results in Example 4, it was intended to verify the diabetes mellitus treatment effect of the GLP-1/protamine-SMCC-Fc complex according to the present invention through an in vivo experiment. For this purpose, a BKS.Cg+/+Leprdb/db mouse model, which is a type 2 diabetes mellitus model, was used, and the mouse is a genetically modified mouse so that it is a model having characteristics in which the insulin value starts to increase from 10 to 14 days after birth, obesity begins and hyperlipidemia appears at 4 to 5 weeks, and from 10 weeks on, a diabetes (glucosuria) positive rate of 100% is exhibited. For the experiment, the complex was orally administered to the mouse model at intervals of 4 days, PBS was administered to the control, and non-fasting blood sugar levels were measured every day until day 32 over time.

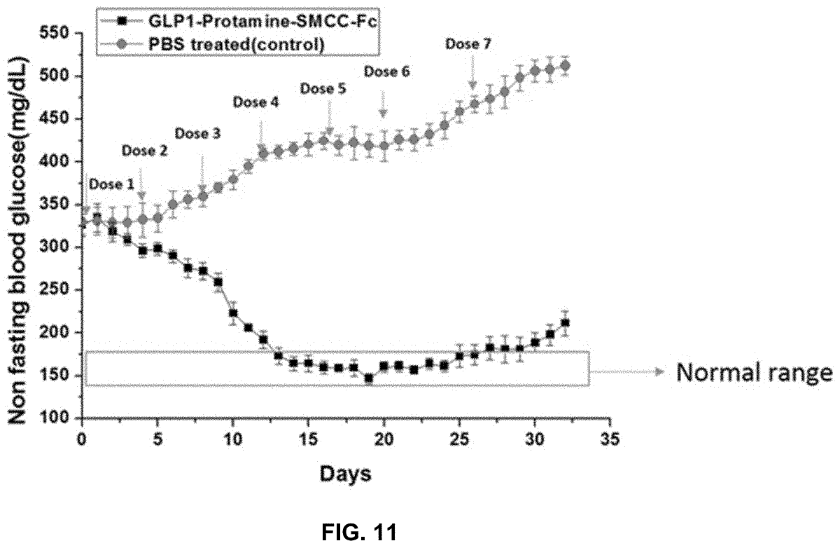

As a result, as illustrated in FIG. 11, it was confirmed that when GLP-1-protamine-SMCC-Fc was administered, the non-fasting blood sugar level dropped to a normal range unlike the control, and through this, it can be seen that the oral administration of the complex according to the present invention has an effect of controlling blood sugar to a normal level.

The above-described description of the present invention is provided for illustrative purposes, and those skilled in the art to which the present invention pertains will understand that the present invention can be easily modified into other specific forms without changing the technical spirit or essential features of the present invention. Therefore, it should be understood that the above-described embodiments are only exemplary in all aspects and are not restrictive.

### INDUSTRIAL APPLICABILITY

The orally-administered gene carrier according to the present invention can stably deliver a gene to the small intestine by protecting the gene from the in vivo environment, and an effect of regulating blood sugar using a complex in which a GLP-1 gene is loaded into the gene carrier is confirmed, so that the complex may be usefully utilized in the field of preventing diabetes mellitus, or developing a therapeutic agent.

What is claimed is:

1. An orally-administered gene carrier comprising:
protamine which binds to a target gene;
an immunoglobulin Fc region; and
a linker which links the protamine and the immunoglobulin Fc region.

2. The gene carrier of claim 1, wherein the immunoglobulin Fc region comprises an amino acid sequence of SEQ ID NO: 1.

3. The gene carrier of claim 1, wherein the immunoglobulin Fc region comprises a base sequence of SEQ ID NO: 2.

4. The gene carrier of claim 1, wherein the immunoglobulin Fc region is derived from any one selected from the group consisting of IgG, IgA, IgD, IgE, and IgM.

5. The gene carrier of claim 4, wherein the immunoglobulin Fc region is derived from IgG.

6. The gene carrier of claim 1, wherein the linker is succinimidyl 4-(N-maleimido-methyl)cyclohexane-1-carboxylate) (SMCC).

7. The gene carrier of claim 1, wherein the protamine-SMCC-Fc gene carrier is prepared by being mixed with the target gene at a weight ratio (w/w) of 1:5 to 150:1.

8. A method for preparing the gene carrier of claim 1, the method comprising the following steps:
(a) preparing a protamine-succinimidyl 4-(N-maleimido-methyl)cyclohexane-1-carboxylate) (SMCC) solution by adding a protamine solution to a SMMC solution;
(b) preparing a protamine-SMCC-Fc solution by adding a Cys-Fc solution to the protamine-SMCC solution; and
(c) obtaining a gene carrier by freeze-drying the prepared protamine-SMCC-Fc solution.

9. A method of treating diabetes mellitus, comprising:
administering to a subject in need thereof an effective amount of a pharmaceutical comprising the gene carrier of claim 1 and a glucagon like peptide-1 (GLP-1) gene bound to the carrier.

10. The method of claim 9, wherein the diabetes mellitus is type 2 diabetes mellitus.

11. The method of claim 9, wherein the GLP-1 comprises a base sequence of SEQ ID NO: 3.

12. The method of claim 9, wherein the pharmaceutical composition is orally administered.

13. (canceled)


SEQUENCE LISTING (3 sequences)

SEQ ID NO: 1 - protein, 73 aa, Artificial Sequence
  hIgG1-Fc
MKWVTFISLLFLFSYSMFWCGGHDLRLPRKKEMSFEWHLFSSTVVPLQKSTRHYVLRDVLVMCVFSERDRGPF

SEQ ID NO: 2 - DNA, 219 bp, Artificial Sequence
  hIgG1-Fc
atgaagtgggtgaccttcatctccctgctgtttctgttctcctactccatgttctggtgcggagggcacgacctgaggctgccgaggaagaaggagatgtcgttcgagtggcacctgttctcgtccaccgtcgtccccttgcagaagagtacgaggcactacgtactccgagacgtgttggtgatgtgcgtcttctcggagagggacagaggcccattt

SEQ ID NO: 3 - DNA, 114 bp, Artificial Sequence
  GLP-1
atgcgtcaacgtcgtcatgctgaagggacctttaccagtgatgtaagttcttatttggaaggccaagctgccaaggaattcattgcttggctggtgaaaggccgatagtctaga